(19) United States
(12) Patent Application Publication
(10) Pub. No.: US 2011/0015589 A1
(43) Pub. Date: Jan. 20, 2011

(54) DISPOSABLE THERAPEUTIC DEVICE

(76) Inventors: Pal Svedman, Malmö (SE); David M. Tumey, Germantown, MD (US); Tianning Xu, Duluth, GA (US); Richard C. Vogel, Gaithersburg, MD (US); Mark S. Meents, Germantown, MD (US)

Correspondence Address: A PATENT LAWYER CORP, PLC;R WILLIAM GRAHAM, 22 S ST CLAIR ST, DAYTON, OH, 45402, US

(21) Appl. No.: 12/502,740

(22) Filed: Jul. 14, 2009

(51) Int. Cl. A61M 1/00

(52) U.S. Cl. 604/313

(57) ABSTRACT

A disposable therapeutic device for the promotion of wound healing, providing fluid irrigation and vacuum drainage of a wound includes a housing containing a controller and fluid moving device in a waterproof manner, the fluid moving means capable of raising, compressing, or transferring fluid, the controller equipped to restrict fluid moving device in accordance with a predetermined treatment plan or duration, a chargeable power source removably connected to the housing, an optional therapeutic member of a compressible dressing or inflatable cuff to provide hemostasis, an identification member for regulating the operation of the device in accordance with a predetermined treatment plan, a disposable container, a pressure sensor and a control display panel.

### BACKGROUND

1. Field of Invention

The invention is generally directed to a disposable therapeutic device for the promotion of wound healing. More particularly, the present invention relates to providing fluid irrigation and vacuum drainage of a wound.

2. Related Art

These devices are normally used in clinical settings such as hospitals or extended care facilities, but patients can often be located in non-clinical environments, where portability, ease of use, and control of therapy parameters is necessary. Such places can, for example, include the home, office or motor vehicles, and at the extreme, military battlefields and other locations where electrical power may be unreliable or unavailable.

Negative pressure wound therapy (NPWT), also known as vacuum drainage or closed-suction drainage, is known. A vacuum source is connected to a semi-occluded or occluded therapeutic member, such as a compressible wound dressing. Various porous dressings comprising gauze, felts, foams, beads and/or fibers can be used in conjunction with an occlusive semi-permeable cover and a controlled vacuum source. In addition to negative pressure, there exist pump devices configured to supply positive pressure to another therapeutic member, such as an inflatable cuff for various medical therapies.

In addition to using negative pressure wound therapy, many devices employ concomitant wound irrigation. For example, a known wound healing apparatus includes a porous dressing made of polyurethane foam placed adjacent a wound and covered by a semi-permeable and flexible plastic sheet. The dressing further includes fluid supply and fluid drainage connections in communication with the cavity formed by the cover, foam and skin. The fluid supply is connected to a fluid source that can include an aqueous topical anesthetic or antibiotic solution, isotonic saline, or other medicaments for use in providing therapy to the wound. The fluid drainage can be connected to a vacuum source where fluid can be removed from the cavity and subatmospheric pressures can be maintained inside the cavity. The wound irrigation apparatus, although able to provide efficacious therapy, is somewhat cumbersome, difficult to use without trained professional medical personnel, and generally impractical outside the clinical setting. Such a device does not address various factors concerning patients outside clinical settings.

Some devices use vacuum sealing of wound dressings consisting of polyvinyl alcohol foam cut to size and stapled to the margins of the wound. Such dressings are covered by a semi-permeable membrane while suction and fluid connections are provided by small plastic tubes which are introduced into the foam generally through the patient's skin. Such devices alternate in time between vacuum drainage and the introduction of aqueous medicaments to the wound site, but do not do both simultaneously. While the prior devices have proven to be useful in fixed therapeutic sites, such devices require improvement to render broader and friendlier use.

### SUMMARY OF THE INVENTION

It is an object to improve wound healing.

It is another object to improve devices for use in treating wounds.

It is an object to improve a pump for use in treating wounds.

It is yet another object to provide a therapeutic device for treating wounds which has improved portability.

It is yet another object to provide a therapeutic device for treating wounds which has improved ease of use.

It is yet another object to provide a therapeutic device for treating wounds which is equipped for predetermined and/or remote control of therapy parameters of time and pressure.

Thus, another object is to provide an improved therapeutic device which is equipped to deliver negative or positive pressure to a wound site.

One embodiment of the invention is directed to a disposable therapeutic device, which includes fluid moving means for one of raising, compressing, or transferring fluid, a therapeutic member operably connected to the fluid moving means and actuated thereby, the therapeutic member operably disposably used on a patient in a manner to deliver therapy to the patient as function of actuation of the fluid moving means; and control means operably associated with the fluid moving means for controlling operation thereof in a manner to restrict use of the fluid moving means by the patient in accordance with a predetermined treatment plan or duration and render the pump inoperable. A chargeable power source to supply power to the fluid moving means and the control means is provided.

More particularly, a wound irrigation system can use a fluid moving means, such as a diaphragm or piston-type pump, to raise, compress and transfer fluid in an electromechanical vacuum apparatus that includes a control means, such as a microprocessor-based device, having stored thereon software configured to control the electromechanical vacuum apparatus, and including one of a timer, means for remote control of the system, and means to restrict the operation of the apparatus to a predetermined treatment plan or duration.

A first vacuum pump can be electrically associated with the microcontroller and capable of generating a vacuum. An optional second vacuum pump is electrically associated with the microcontroller and is capable of maintaining a predetermined vacuum level. A first electronic vacuum-pressure sensor can be operably associated with the vacuum pump(s) and the microcontroller for monitoring vacuum level.

A fluid-tight wound exudate collection canister can be provided and can include an integrated barrier, such as a float valve, porous polymer filter or hydrophobic filter, to prevent contents from escaping the canister. Single-lumen tubing can be associated with the canister and vacuum pump(s) for communicating vacuum pressure therefrom. A second electronic vacuum-pressure sensor can be operably associated with the canister and the microcontroller for monitoring canister vacuum.

A dressing includes a porous material and semi-permeable flexible cover. Single-lumen tubing is associated with the dressing and the canister to communicate vacuum pressure therefrom. An irrigation vessel can be provided to contain a fluid to be used in irrigating the wound. Single-lumen tubing is associated with the irrigation vessel and the dressing to communicate fluid thereto.

The electromechanical vacuum apparatus housing may incorporate a compartment that can hold the irrigation vessel. The electromechanical vacuum apparatus can preferably include a device for regulating the quantity of fluid flowing from said irrigation vessel to said dressing. This device can comprise a mechanical or pneumatically actuated valve or clamp.

The electromechanical vacuum apparatus may include commercially available disposable storage batteries enabling portable operation thereof. Alternative power sources include rechargeable or reprocessable batteries which are removably connected to a housing, which contains the fluid moving means and control means, both of which require power in a waterproof environment. Other alternative power sources are solar energy, a manually operated generator in combination with a storage device such as a supercapacitor, or a pneumatic accumulator.

An embodiment of the invention includes a method for improving the generation and control of a therapeutic vacuum. In this embodiment, a multi-modal algorithm monitors pressure signals from a first electronic vacuum-pressure sensor associated with a vacuum pump and capable of measuring the output pressure from the pump. The algorithm further monitors pressure signals from a second electronic vacuum-pressure sensor associated with a collection canister and capable of measuring the subatmospheric pressure inside the canister. The second electronic vacuum-pressure sensor may also be associated with the wound dressing and capable of measuring the subatmospheric pressure inside the dressing. The canister is connected to the vacuum pump by a single-lumen tube that communicates subatmospheric pressure therefrom. The canister is connected to a suitable dressing by a single-lumen tube that communicates subatmospheric pressure thereto.

At the start of therapy, both the first and second electronic vacuum-pressure sensors indicate the system is equilibrated at atmospheric pressure. A first-mode control algorithm is employed to rapidly remove the air in the canister and dressing, and thus create a vacuum. The first-mode implemented by the control algorithm is subsequently referred to herein as the “draw down” mode. Once the subatmospheric pressure in the canister and dressing have reached a preset threshold as indicated by the first and second electronic vacuum-pressure sensors respectively, the algorithm employs a second-mode that maintains the desired level of subatmospheric pressure in both the canister and the dressing for the duration of the therapy. The second-mode implemented by the control algorithm is subsequently referred to herein as the “maintenance” mode.

The second-mode control algorithm is configured to operate the vacuum pump at a reduced speed thus minimizing unwanted mechanical noise. In an alternative embodiment, a second vacuum pump can be used for the maintenance mode, which has a reduced capacity, is smaller, and produces significantly lower levels of unwanted mechanical noise. The second-mode control algorithm is configured to permit the maintenance of vacuum in the presence of small leaks, which invariably occur at the various system interfaces and connection points. The method can be performed by, for example, a microprocessor-based device.

The control means can be provided with a timer for restricting the use as a function of a predetermined time. Alternatively, an identification member can be provided with the device such that the control means restricts use as a function of the identification member. The control means may include a Radio Frequency Identification Chip (RFID) chip available under the trademark Omni-ID™. The control means can be operably associated with a remote control for restricting the use of the device.

### BRIEF DESCRIPTION OF THE DRAWINGS

FIG. 1 is a schematic illustrating the device of the invention.

FIG. 1A depicts a part of the invention.

### DETAILED DESCRIPTION

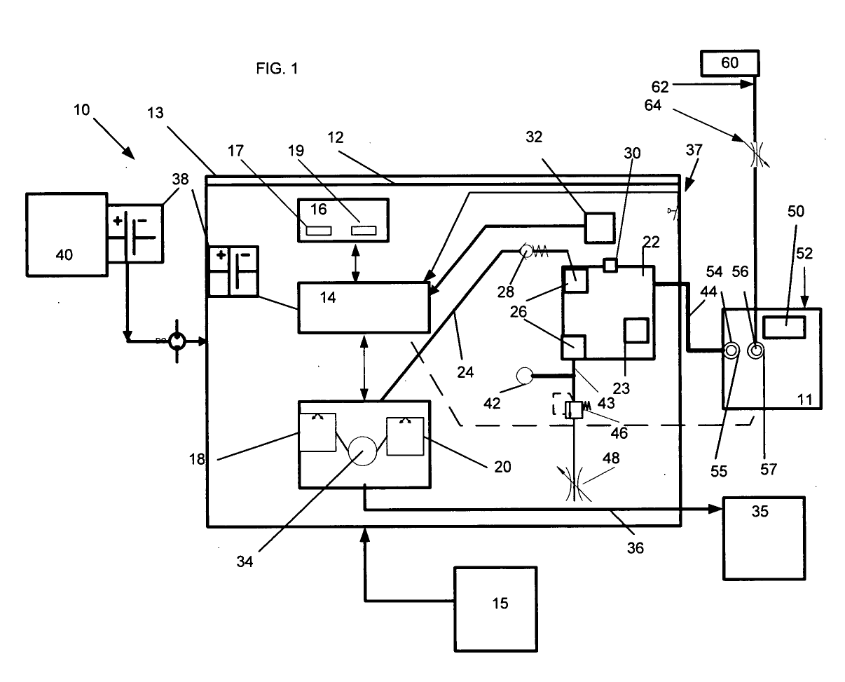

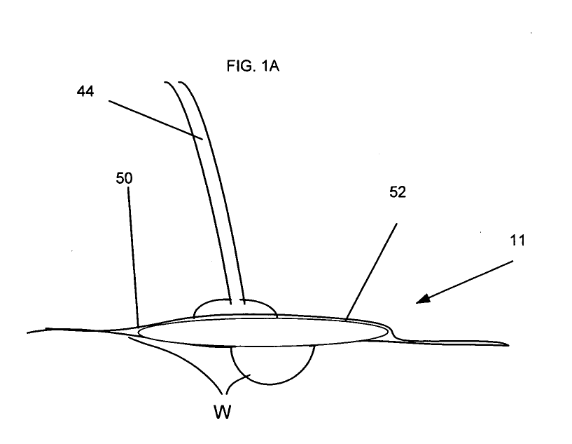

As illustrated in FIG. 1, a disposable therapeutic device of the instant invention is generally designated by the numeral 10. The disposable therapeutic device 10 can preferably include a housing 12 which provides an improved therapeutic device with multiple uses and portability. The housing 12 can preferably be formed in a waterproof manner to protect components therein. In this regard, housing 12 can have a watertight sealed access panel 13 through which components can be accessed.

The device 10 can include a processor 14, which can be a microcontroller having an embedded microprocessor, Random Access Memory (RAM) and Flash Memory (FM). FM can preferably contain the programming instructions for a control algorithm. FM can preferably be non-volatile and retains its programming when the power is terminated. RAM can be utilized by the control algorithm for storing variables such as pressure measurements, alarm counts and the like, which the control algorithm uses while generating and maintaining the vacuum.

A membrane keypad and a light emitting diode LED or liquid crystal display (LCD) 16 can be electrically associated with processor 14 through a communication link, such as a cable. Keypad switches provide power control and are used to preset the desired pressure/vacuum levels. Light emitting diodes 17, 19 can be provided to indicate alarm conditions associated with canister fluid level, leaks of pressure in the dressing and canister, and power remaining in the power source.

Microcontroller 14 is electrically associated with, and controls the operation of, a first vacuum pump 18 and an optional second vacuum pump 20 through electrical connections. First vacuum pump 18 and optional second vacuum pump 20 can be one of many types including, for example, the pumps sold under the trademarks Hargraves® and Thomas®. Vacuum pumps 18 and 20 can use, for example, a reciprocating diaphragm or piston to create vacuum and can be typically powered by a direct current (DC) motor that can also optionally use a brushless commutator for increased reliability and longevity. Vacuum pumps 18 and 20 can be pneumatically associated with a disposable exudate collection canister 22 through a single-lumen tube 24.

In one embodiment, canister 22 has a volume which does not exceed 1000 ml. This can prevent accidental exsanguination of a patient in the event hemostasis has not yet been achieved at the wound site. Canister 22 can be of a custom design or one available off-the-shelf and sold under the trademark DeRoyal®.

In addition, a fluid barrier 26, which can be a back flow valve or filter, is associated with canister 22 and is configured to prevent fluids collected in canister 22 from escaping into tubing 24 and fouling the vacuum return path. Barrier 26 can be of a mechanical float design or may have one or more membranes of hydrophobic material such as those available under the trademark GoreTex™. Barrier 26 can also be fabricated from a porous polymer such as that which is available under the trademark MicroPore™. A secondary barrier 28 using a hydrophobic membrane or valve is inserted in-line with pneumatic tubing 24 to prevent fluid ingress into the system in the event barrier 26 fails to operate as intended. Pneumatic tubing 24 can connect to first vacuum pump 18 and optional second vacuum pump 20 through “T” connectors.

An identification member 30, such as radio frequency identification (RFID) tag, can be physically associated with the canister 22 and an RFID sensor 32 operably associated with the microcontroller 14 such that the microcontroller 14 can restrict use of the device 10 to a predetermined canister 22. Thus, if a canister 22 does not have a predetermined RFID chip, the device 10 will not operate. Another embodiment envisions software resident on microcontroller 14 which restricts the use of the device 10 to a predetermined time period such as 90 days for example. In this way, the patient using the device 10 may use the device 10 for a prescribed time period and then the device 10 automatically times out per a particular therapeutic plan for that patient. This also enables a reminder of the time and date for the next dressing change or physician appointment. It is also contemplated that the microcontroller 14 be operably provided with a remote control 15 and communication link, such as a transceiver, wherein the device 10 can be shut down remotely when a particular therapeutic plan for that patient has ended. Likewise, remote control 15 can be utilized to provide additional time after the therapeutic device times out.

Vacuum-pressure sensor 34 is pneumatically associated with first vacuum pump 18 and optional vacuum pump 20 and electrically associated with microcontroller 14. Pressure sensor 34 provides a vacuum-pressure signal to the microprocessor enabling a control algorithm to monitor vacuum pressure at the outlet of the vacuum pumps 18 and 20.

An acoustic muffler can be provided and pneumatically associated with the exhaust ports of vacuum pumps 18 and 20 and configured to reduce exhaust noise produced by the pumps during operation. In normal operation of device 10, first vacuum pump 18 can be used to generate the initial or “draw-down” vacuum while optional second vacuum pump 20 can be used to maintain a desired vacuum within the system compensating for any leaks or pressure fluctuations. Vacuum pump 20 can be smaller and quieter than vacuum pump 18 providing a means to maintain desired pressure without disturbing the patient. It is contemplated by the instant invention that pumps 18 and 20 can also be employed to create a positive pressure for purposes of applying pressure to an inflatable member 35, such as a cuff or pressure bandage, through tubing 36. A switch 37 can be operatively disposed on housing 12 in operable connection with microcontroller 14 to enable selection of positive and negative pressure from pumps 18/20.

One or more battery(ies) 38 can preferably be provided to permit portable operation of the device 10. Battery 38 can be Lithium Ion (LiIon), Nickel-Metal-Hydride (NiMH), Nickel-Cadmium, (NiCd) or their equivalent, and can be electrically associated with microcontroller 14 through electrical connections. Battery 38 can be of a rechargeable type which is preferably removably disposed in connection with the housing 12 and can be replaced with a secondary battery 38 when needed. A recharger 40 is provided to keep one battery 38 charged at all times. Additionally, it is contemplated that the device 10 can be equipped to be powered or charged by recharger 40 or by circuits related with microcontroller 14 if such source of power is available. When an external source of power is not available and the device 10 is to operate in a portable mode, battery 38 supplies power to the device 10. The battery 38 can be rechargeable or reprocessable and can preferably be removably stored in a waterproof manner within housing 12 which also likewise contains the pumps 18, 20 and microcontroller 14.

A second pressure sensor 42 is pneumatically associated with canister 22 through a sensor port 43. Pressure sensor 42 can be electrically associated with microcontroller 14 and provides a vacuum-pressure signal to microprocessor enabling control algorithm to monitor vacuum pressure inside canister 22 and dressing 11. A “T” connector can be connected to port 43, to pressure sensor 42 and a vacuum-pressure relief solenoid 46 configured to relieve pressure in the canister 22 and dressing 11 in the event of an alarm condition, or if power is turned off. Solenoid 46, can be, for example, one available under the trademark Parker Hannifin® or Pneutronics®; Solenoid 46 is electrically associated with, and controlled by, microprocessor of microcontroller 14. Solenoid 46 can be configured to vent vacuum pressure to atmosphere when an electrical coil associated therewith is de-energized as would be the case if the power is turned off. An orifice restrictor 48 may optionally be provided in-line with solenoid 46 and pneumatic tube 44 to regulate the rate at which vacuum is relieved to atmospheric pressure when solenoid 46 is de-energized. Orifice restrictor 48 is, for example, available under the trademark AirLogic®.

A wound dressing 11 can preferably include a sterile porous substrate 50, which can be a polyurethane foam, polyvinyl alcohol foam, gauze, felt or other suitable material, a semi-permeable adhesive cover 52 such as that sold under the trademark DeRoyal® or Avery Denison®, an inlet port 56 and a suction port 54. Substrate 50 is configured to distribute vacuum pressure evenly throughout the entire wound bed and has mechanical properties suitable for promoting the formation of granular tissue and approximating the wound margins.

In addition, when vacuum is applied to dressing 11, substrate 50 creates micro- and macro-strain at the cellular level of the wound stimulating the production of various growth factors and other cytokines, and promoting cell proliferation. Dressing 11 is fluidically associated with canister 22 through single-lumen tube 44. The vacuum pressure in a cavity formed by substrate 50 of dressing 11 is largely the same as the vacuum pressure inside canister 22 minus the weight of any standing fluid inside tubing 44.

A fluid vessel 60, which can be a standard IV bag, contains medicinal fluids such as aqueous topical antibiotics, analgesics, physiologic bleaches, or isotonic saline. Fluid vessel 60 is removably connected to dressing 11 though port 56 and single-lumen tube 62.

An optional flow control device 64 can be placed in-line with tubing 62 to permit accurate regulation of the fluid flow from vessel 60 to dressing 11. In normal operation, continuous wound site irrigation is provided as treatment fluids move from vessel 60 through dressing 11 and into collection canister 22. This continuous irrigation keeps the wound clean and helps to manage infection. In addition, effluent produced at the wound site and collected by substrate 50 will be removed to canister 22 when the system is under vacuum.

The device 10 is particularly well suited for providing therapeutic wound irrigation and vacuum drainage and provides for a self-contained plastic housing configured to be worn around the waist or carried in a pouch over the shoulder for patients who are ambulatory, and hung from the footboard or headboard of a bed for patients who are non-ambulatory. Membrane keypad and display 16 is provided to enable the adjustment of therapeutic parameters and to turn the unit on and off.

Depressing the power button on membrane switch 16 will turn the power to device 10 on/off. While it is contemplated that the membrane switch 16 be equipped with keys to adjust therapeutic pressure up and down, the microcontroller 14 can preferably be equipped to control the pressure in accordance with sensed pressure and condition to maintain pressure in an operable range between −70 mmHg and −150 mmHg with a working range of between 0 and −500 mmHg, for example. Although these pressure settings are provided by way of example, they are not intended to be limiting because other pressures can be utilized for wound-type specific applications. The membrane 16 can also be equipped with LED 17 to indicate a leak alarm and/or LED 19 indicates a full-canister alarm. When either alarm condition is detected, these LEDs will light in conjunction with an audible chime which is also included in the device 10.

Housing 12 can incorporate a compartment configured in such a way as to receive and store a standard IV bag 60 or can be externally coupled to thereto. IV bag 60 may contain an aqueous topical wound treatment fluid that is utilized by the device 60 to provide continuous irrigation. A belt clip can provided for attaching to a patient's belt and an optional waist strap or shoulder strap is provided for patients who do not or cannot wear belts.

Canister 22 is provided for exudate collection and can preferably be configured as currently known in the field with a vacuum-sealing means and associated fluid barrier 26, vacuum sensor port 43 and associated protective hydrophobic filter, contact-clear translucent body, clear graduated measurement window, locking means and tubing connection means. Collection canister 22 typically has a volume less than 1000 ml to prevent accidental exsanguination of a patient if hemostasis is not achieved in the wound. Fluid barriers 26 can be, for example, those sold under the trademark MicroPore® or GoreTex® and ensure the contents of canister 22 do not inadvertently ingress into pumps 18, 20 of housing 12 and subsequently cause contamination of thereof.

Pressure sensor 42 enables microcontroller 14 to measure the pressure within the canister 22 as a proxy for the therapeutic vacuum pressure under the dressing 11. Optionally, tubing 62 can be multilumen tubing providing one conduit for the irrigation fluid to travel to dressing 11 and another conduit for the vacuum drainage. Thus, IV bag 60, tubing 62, dressing 11 and canister 22 provide a closed fluid pathway. In this embodiment, canister 22 would be single-use disposable and may be filled with a solidifying agent 23 to enable the contents to solidify prior to disposal. Solidifying agents are available, for example, under the trademark DeRoyal® and Isolyzer®. The solidifying agents prevent fluid from sloshing around inside the canister particularly when the patient is mobile, such as would be the case if the patient were travelling in a motor vehicle. In addition, solidifying agents are available with antimicrobials that can destroy pathogens and help prevent aerosolization of bacteria.

At the termination of optional multilumen tubing 62, there can be provided a self-adhesive dressing connector 57 for attaching the tubing to drape 52 with substantially air-tight seal. Dressing connector 11 can have an annular pressure-sensitive adhesive ring with a release liner that is removed prior to application. Port 56 can be formed as a port cut in drape 52 and dressing connector 57 would be positioned in alignment with said port. This enables irrigation fluid to both enter and leave the dressing through a single port. In an alternative embodiment, tube 62 can bifurcate at the terminus and connect to two dressing connectors 57 which allow the irrigation port to be physically separated from the vacuum drainage port thus forcing irrigation fluid to flow though the entire length of the dressing if it is so desired. Similarly, port 54 and connector 55 can be provided to connect optional multilumen tubing 44 to dressing 11. In this arrangement, the second lumen may be used to directly measure the pressure in dressing 11.

Fluid vessel 60 can be of the type which includes a self-sealing needle port situated on the superior aspect of the vessel 60 and a regulated drip port situated on the inferior aspect of the vessel. The needle port permits the introduction of a hypodermic needle for the administration of aqueous topical wound treatment fluids. These aqueous topical fluids can include a topical anesthetic such as Lidocaine, antibiotics such as Bacitracin or Sulfamide-Acetate; physiologic bleach such as Chlorpactin or Dakins solution; and antiseptics such as Lavasept or Octenisept. Regulated drip port permits fluid within vessel 60 to egress slowly and continuously into porous substrate 50 whereupon the therapeutic benefits can be imparted to the wound site. Single-lumen drainage tube 44 provides enough vacuum to keep the dressing 11 at sub-atmospheric pressure and to remove fluids, which include the irrigation fluid and wound exudates. With this modification, the need for an external fluid vessel and associated tubing and connectors can be eliminated making the dressing more user friendly for patient and clinician alike.

In typical clinical use of this alternate embodiment, dressing 11 is applied to the wound site by first cutting porous substrate 50 to fit the margins of the wound. Next, semi-permeable drape 52 is attached and sealed over the dressing and periwound. A hole approximately ⅜″ diameter can be made in drape 52 central to porous substrate 50. Fluid vessel 60 is attached by adhesive annular ring 57 with port 56 aligned with the hole previously cut in drape 52. Once the fluid vessel 60 is hermitically sealed to the drape 52, a properly prepared hypodermic needle is inserted in self-sealing needle port and fluid vessel 60 subsequently filled with the desired aqueous topical wound treatment solution.

For the majority of applications, the technique for providing therapeutic wound irrigation and vacuum drainage is illustrated. The single lumen drainage tube 44 is provided for the application of vacuum and removal of fluids from the wound site. Fluid vessel 60 can be situated outside and superior to semi-permeable substrate 50. An annular adhesive ring 57 is provided on port 56 for attachment of single-lumen irrigation tubing 62 to drape 52. Similarly, a needle port permits the introduction of a hypodermic needle for the administration of aqueous topical wound treatment fluids as described above, for example, a caregiver may want to add a topical antibiotic to a bag of isotonic saline. Adjustable optional flow control device 64 permits fluid within vessel 60 to egress slowly and continuously into porous substrate 50 through hole 56 in drape 52 whereupon the therapeutic benefits can be imparted to the wound site. Single-lumen drainage tube 44 provides enough vacuum to keep the dressing 11 at sub-atmospheric pressure and to remove fluids which include the irrigation fluid and wound exudates.

Because of the potential chemical interactions between the various materials used in the construction of dressing 11, attention must be paid to the types of aqueous topical wound fluids used to ensure compatibility. The above described embodiments are set forth by way of example and are not limiting. It will be readily apparent that obvious modifications, derivations and variations can be made to the embodiments. For example, the vacuum pumps described having either a diaphragm or piston-type could also be one of a syringe based system, bellows, or even an oscillating linear pump. Accordingly, the claims appended hereto should be read in their full scope including any such modifications, derivations and variations.

What is claimed is:

1. A disposable therapeutic device, which includes:
fluid moving means for one of raising, compressing, or transferring fluid;
a therapeutic member operably connected to said fluid moving means and actuated thereby, said therapeutic member operably disposably used on a patient in a manner to deliver therapy to the patient as function of actuation of said fluid moving means; and
control means operably associated with said fluid moving means for controlling operation thereof in a manner to restrict use of said fluid moving means by said patient in accordance with a predetermined treatment plan or duration and render inoperable said device.

2. The disposable therapeutic device of claim 1, which includes a chargeable power source to supply power to said fluid moving means.

3. The disposable therapeutic device of claim 2, wherein said chargeable power source is removable.

4. The disposable therapeutic device of claim 2, wherein said chargeable power source is a battery.

5. The disposable therapeutic device of claim 4, wherein said chargeable power source is removable.

6. The disposable therapeutic device of claim 1, wherein said therapeutic member includes a compressible dressing.

7. The disposable therapeutic device of claim 1, wherein said therapeutic member includes an inflatable cuff.

8. The disposable therapeutic device of claim 1, wherein said control means includes a timer for restricting said use as a function of a predetermined time.

9. The disposable therapeutic device of claim 1, which further includes an identification member such that said control means restricts use as a function of a said identification member.

10. The disposable therapeutic device of claim 1, wherein said control means includes means for remote control thereof for restricting said use.

11. The disposable therapeutic device of claim 1, which further includes a disposable container removably operably interconnected to said fluid moving means and to said therapeutic member to receive waste fluid therein as a result of actuation of said fluid moving means.

12. The disposable therapeutic device of claim 1, which further includes a housing operably containing said control means and said fluid moving means.

13. The disposable therapeutic device of claim 12, which further includes a chargeable power source operably connected to said housing to supply power to said fluid moving means.

14. The disposable therapeutic device of claim 13, wherein said chargeable power source is removably connected to said housing.

15. The disposable therapeutic device of claim 12, wherein said housing is further characterized to contain said control means and said fluid moving means in a waterproof manner.

16. The disposable therapeutic device of claim 1, which further includes a pressure sensor operably connected to said control means and said therapeutic member such that said control means controls said fluid moving means as a function of said sensed pressure.

17. The disposable therapeutic device of claim 12, wherein said housing includes a control display panel operably thereon and connected to said control means.

18. A therapeutic device of the type which includes fluid moving means for one of raising, compressing, or transferring fluid, a therapeutic member operably connected to said fluid moving means and actuated thereby, the therapeutic member operably disposable for use on a patient in a manner to deliver therapy to the patient as function of actuation of said fluid moving means, wherein the improvement includes:
a chargeable power source operably connected to the fluid flowing means.

19. The therapeutic device of claim 18, which includes control means operably associated with said fluid moving means for controlling operation thereof in a manner to restrict use of said fluid moving means by said patient in accordance with a predetermined treatment.

20. The therapeutic device of claim 18, wherein said chargeable power source is removable.

21. The therapeutic device of claim 18, wherein said therapeutic member includes a compressible dressing.

22. The therapeutic device of claim 18, wherein said therapeutic member includes an inflatable cuff.

23. The therapeutic device of claim 19, wherein said control means includes a timer for restricting said use as a function of a predetermined time.

24. The therapeutic device of claim 19, which further includes an identification member such that said control means restricts use as a function of a said identification member.

25. The therapeutic device of claim 19, wherein said control means includes means for remotely control thereof for restricting said use.

26. The therapeutic device of claim 18, which further includes a disposable container removably operably interconnected to said fluid moving means and to said therapeutic member to receive waste fluid therein as a result of actuation of said fluid moving means.

27. The therapeutic device of claim 19, which further includes a housing operably containing said control means and said fluid moving means.

28. The therapeutic device of claim 27, wherein said chargeable power source is operably connected to said housing to supply power to said fluid moving means.

29. The therapeutic device of claim 28, wherein said chargeable power source is removably connected to said housing.

30. The therapeutic device of claim 27, wherein said housing is further characterized to contain said control means and said fluid moving means in a waterproof manner.

31. The therapeutic device of claim 19, which further includes a pressure sensor operably connected to said control means and said therapeutic member such that said control means controls said fluid moving means as a function of said sensed pressure.

32. The therapeutic device of claim 27, wherein said housing includes a control display panel operably thereon and connected to said control means.
